(19) United States
(12) Patent Application Publication
(10) Pub. No.: US 2004/0107057 A1
(43) Pub. Date: Jun. 3, 2004

(54) Enhanced protein separation and analysis

(76) Inventors: Roderick A Capaldi, Eugene, OR (US); Wayne F Patton, Eugene, OR (US)

Correspondence Address: KLARQUIST SPARKMAN, LLP, 121 SW SALMON STREET, SUITE 1600, PORTLAND, OR, 97204, US

(21) Appl. No.: 10/472,581

(22) PCT Filed: Mar. 22, 2002
(86) PCT No.: PCT/US02/08723

(51) Int. Cl. G06F 19/00; G01N 33/48; C07K 1/00; G06G 7/48; A01N 37/18

(52) U.S. Cl. 702/27; 702/19; 703/11; 530/344; 514/2

(57) ABSTRACT

Methods for enhancing separation and analysis of biological molecules, particularly proteins, and for characterizing tissue, cell, and subcellular (e.g., organelle) expressed protein profiles (proteomes or protein fingerprints) are disclosed. Multi-dimensional diagrams that illustrate the characteristics of the proteins in a sample, based at least in part on interactions between proteins in the system can be produced. In certain embodiments, the diagrams are three-dimensional and incorporate information on protein-protein interactions, protein charge, and protein size for substantially all of the protein species in the sample. Also described are methods of using the provided multi-dimensional diagrams to detect changes in biological systems that are for instance due to disease, drug treatment, environmental condition, and so forth. Methods are provided for correlating changes in three-dimensional proteomic diagrams to disease diagnosis and prognosis, toxicology, therapeutic compound (e.g., drug or hormone) efficacy and mode of action, and drug design.

### STATEMENT OF GOVERNMENTAL INTEREST

This work was supported by funds from the National Institutes of Health (NIH), under Heart and Lung grant number 24526. The government has certain rights in this invention.

### FIELD

This disclosure relates to the field of proteomics, and particularly to enhanced protein separation techniques useful in the study of proteomes.

### BACKGROUND

The mitochondrion is one of the most complex as well as one of the most important organelles in a eukaryotic cell. It consists of multiple compartments (Frey and Mannella, TIBS, 25:319-324, 2000; Perkins et al., J. Bioenerg. Biomembr., 30:431-442, 1998; Perkins et al., J. Struct. Biol., 119:260-272, 1997) containing a vast number of proteins which must somehow be arranged to carry out a variety of processes fundamental to cell function. These processes include heme synthesis, the TCA cycle, β-oxidation of fatty acids, the urea cycle, electron transport, and oxidative phosphorylation. Electron transport and oxidative phosphorylation alone require the coordinated action of five enzyme complexes, which together are comprised of an estimated 86 different structural proteins (Saraste, Science, 283:1488-1493, 1999). In addition, there are non-structural proteins which are required for the proper assembly and regulation of these complexes (e.g. Surf I and ScoII) (Sue et al., Ann. Neurol., 47:589-595, 2000; Papadopoulou et al., Nat. Genet., 23:333-337, 1999; Tiranti et al., Am. J. Hum. Genet., 63:1609-1621, 1998; Poyau et al., Hum. Genet., 106:194-205, 2000). To add further complexity to this organelle, 13 of the structural proteins are encoded by mitochondrial DNA (Taanman, Biochim. Biophys. Acta, 1410:103-123, 1999).

There is also increasing evidence that mitochondria play an important role in cell death and aging. The importance of mitochondria to apoptosis was first indicated when bcl-2 was identified as a mitochondrial protein that could prevent apoptosis (Hockenbery et a., Nature, 348:334-336, 1990). Since this initial observation, it has further been noted that in a cell-free system, DNA fragmentation was dependent upon a mitochondrial fraction (Newmeyer, Cell, 79:353-364, 1994). In addition, it is now known that during apoptosis, mitochondria release several pro-apoptotic proteins including cytochrome c (Liu et al., Cell, 86:147-157, 1996) and apoptosis inducing factor (Susin et al., J. Exp. Med, 184:1331-1341, 1996). These facts have led to suggestions that mitochondrial dysfunction, by increasing the rate of apoptosis, is critically important in neurodegenerative disorders including Alzheimer's and Parkinson's diseases (Lemasters et al., J. Bioenerg. Biomembr., 31:305-319, 1999; Beal, Trends Neurosci., 23:298-304, 2000).

Due to the fundamental role mitochondria play in cell life and cell death, interest in a mitochondrial proteome map has grown significantly (Scharfe et al., Nucleic Acids Res., 28:155 2000; Rabilloud et al., Electrophoresis, 19:1006-1014, 1998). Such a map would allow researchers to compare the pattern obtained from an altered mitochondrial sample, such as a cell line from a patient with a mitochondrial disease, to a reference map and would provide information about differences in protein expression. Up to now, most attempts to obtain a human mitochondrial 2-D map have involved solubilization of whole mitochondria or even whole cells (Rabilloud et al., Electrophoresis, 19:1006-1014, 1998; Seow et al., Electrophoresis, 21:1787-17813, 2000; Langen et al., Electrophoresis, 20:907-916, 1999). This has led to elaborate two-dimensional (2-D) patterns containing more spots than can be optimally resolved for analysis, particularly as many proteins appear to be present in multiple forms due to post-translational and/or preparative modifications (e.g deamidation). In addition, such maps provide little information about the assembly-state or functionality of individual protein complexes. Furthermore, a disproportionate number of proteins in the mitochondrion are membrane associated making them difficult to solubilize for isoelectric focusing.

There is an ongoing effort in several laboratories to obtain a mitochondrial proteome for use in diagnosis of diseases, to identify targets for drug therapy, and to screen for unwanted drug side effects. The most advanced human mitochondrial proteome has been reported by Rabilloud and colleagues (Electrophoresis, 19:1006-1014, 1998). Their approach has been to resolve placental mitochondrial proteins using the now classical 2-D-gel methodology of isoelectric focusing in the first dimension and SDS-PAGE in the second dimension. However, there are a number of problems with this most straightforward approach. First, the vast number of spots are not optimally separated, particularly as many components appear to be present in multiple forms due to post-translational modification and/or modification occurring during sample preparation. In addition, a considerable number of mitochondrial proteins are small, i.e., MW below 10,000, and these proteins are often difficult to resolve by standard methods. Furthermore, a surprisingly large number of mitochondrial proteins are highly basic (pKs>9.0), and a majority of these proteins are membrane bound. Of the membrane-associated proteins, a high proportion is hydrophobic and difficult to solubilize. Thus, they are not well represented in the proteome of Rabilloud et at (Electrophoresis, 19:1006-1014, 1998), as these authors acknowledge.

In spite of recent advances, current 2-D-PAGE analysis is still inadequate for separating all of the proteins in a system, or even all of the proteins in an organelle. It is to inadequacies in existing separation and analysis techniques that this invention is directed.

### SUMMARY OF THE DISCLOSURE

The inventors have surprisingly found that proteome analysis can be dramatically improved by including a preliminary separation of samples based on their interactions with other proteins (their tertiary structure). Examples of such preliminary separation are sucrose gradients and non-denaturing gel electrophoresis. Using a preliminary separation step that does not fully disrupt the tertiary structure of protein complexes, a third dimension can be added to traditional proteomics analysis. The three separations are based on (A) association of proteins in complexes, (B) isoelectric point, and (C) size. Addition of the preliminary separation (e.g., separation through a sucrose gradient) enables detection of disturbances in protein-protein interactions in a system, such as may be caused by changes in protein expression level, protein confirmation, or post-translational protein modifications, for example. In addition, this preliminary separation step provides the surprising advantage of permitting a higher proportion of hydrophobic proteins to be separated and identified in subsequent analysis steps.

To address the above problems, the inventors have developed a 3-dimensional (3-D) system for analysis of proteomes, such as the mitochondrial proteome. In a preferred embodiment, the first step involves reproducible, discontinuous sucrose gradient separation of detergent-solubilized proteins. The fractions obtained in this step contain protein complexes differentiated by size. These fractions then can be used to measure biologically relevant enzyme activities, to separate proteins by standard SDS-PAGE, and to resolve proteins by 2-D gel electrophoresis (e.g., using IEF in the first dimension followed by SDS-PAGE in the second dimension). This approach greatly enhances the resolution of proteins and further provides functional information about protein complexes within the system.

Provided herein in specific embodiments are methods for creating three-dimensional representations of the protein complement of a biological sample. Examples of these methods include at least three sequential separation phases, wherein the first is a non-denaturing separation (such as a size or buoyant density gradient separation, e.g., sucrose gradient separation, or aqueous 2-phase partitioning, or a non-denaturing agarose gel electrophoresis separation). The resultant separated sample is then divided into a plurality of identifiable sub-fractions, which occur in an identifiable order based on fractionation or other criteria. One or more, or all, of these fractions are then subjected to second and third separation stages.

The second and third phases (which occur subsequent to the first phase but not necessarily in that order) can be separations based on net protein charge (e.g., isoelectric focusing, capillary electrophoresis, or isotachyphoresis) or protein size (e.g., SDS-PAGE, sizing gel, or mass spectroscopy).

The data produced by the sequential separation of the proteins, or representations of this data, then can be assembled into a three-dimensional representation of the proteins in the original sample.

The foregoing and other features and advantages will become more apparent from the following detailed description of several embodiments, which proceeds with reference to the accompanying figures.

### BRIEF DESCRIPTION OF THE FIGURES

FIG. 1 shows a comparison of sucrose gradient fractions, separated by SDS-PAGE, from three different sources of mitochondria: (FIG. 1A) Bovine heart; (FIG. 1B) Human brain; (FIG. 1C) MRC-5 fibroblasts. The fractions were obtained from gradient B with the 35% fraction omitted as described herein. Lanes 1-9 correspond to Fractions 1-9 respectively in each gel. The samples were applied to 10-20% SDS PAGE and stained with SyproRuby™ protein gel stain.

FIG. 2 shows TCA precipitated fractions from bovine heart mitochondria after separation by sucrose gradient centrifugation. 500 μl of each fraction was TCA precipitated and run on an 8-20% polyacrylamide gel to optimize separation of proteins in the molecular weight range between 19-200 kDa. The gels were stained with Comassie Brilliant Blue. (FIG. 2A) Lanes 1-9 correspond to fractions 1-9 respectively as obtained from gradient B. (FIG. 2B) Fraction 1 from gradient A.

FIG. 3 shows a Western blot analysis of mitochondrial proteins from three different sources after separation using sucrose gradient A. The complexes were identified by subunit specific monoclonal antibodies as described herein. (FIG. 3A) Western blot of subunits from the five respiratory chain complexes in sucrose gradient fractions from MRC-5 mitochondria. (FIGS. 3B-3F) Results of a densitometric scan of the gel of (FIG. 3A). Each respiratory chain complex subunit was plotted individually; the darkest intensity for each antibody was set to 100%. Shown are gradients of bovine heart (dotted), MRC-5 fibroblasts (solid) and MRC-5 rho0 (dashed).

FIG. 4 is a graph showing the ATPase (solid line) and creatine kinase (dashed line) activity measurements measured in bovine heart mitochondrial fractions separated on gradient A.

FIG. 5 shows 2-D gels of bovine heart mitochondrial proteins in (FIG. 5A) fraction 3 and (FIG. 5B) fraction 4 after sucrose gradient B. Proteins were separated on IPG strips (3-10 linear) prior to separation on a 10% homogenous SDS-polyacrylamide gel. The gels were stained with SyproRuby™ protein gel stain and imaged using a Fuji FLA3000 scanner. Proteins mainly present in fraction 3 are highlighted with solid boxes, proteins mainly present in fraction 4 are highlighted by circles, and proteins unique in either fraction are highlighted by dashed boxes.

FIG. 6 shows a pictorial model of an example of three dimensional protein separation and the information obtained from each dimension.

### DETAILED DESCRIPTION

I. Abbreviations

2-DE: 2-dimensional electrophoresis

COX: cytochrome oxidase

IEF: iso-electric focusing

IPG: immobilized pH gradients

LM: laurylmaltoside (β-dodecyl maltopyranoside)

mAb: monoclonal antibody

PD: population doubling

PMSF: phenyl methylsulfonylfluoride

SDS: sodium dodecyl sulfate

II. Terms

Unless otherwise noted, technical terms are used according to conventional usage. Definitions of common terms in molecular biology may be found for instance in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechology: a Comprehensive Desk Reference, published by VCH Publishers, Inc., 1995 (ISBN 1-56081-569-8).

In order to facilitate review of the various embodiments, the following explanations of specific terms are provided:

Isolated: An “isolated” biological component (such as a nucleic acid molecule, protein or organelle) has been substantially separated or purified away from other biological components in the cell of the organism in which the component naturally occurs, ie., other chromosomal and extra-chromosomal DNA and RNA, proteins and organelles.

Pharmaceutical/therapeutic agent: Any agent, such as a protein, peptide (e.g. hormone peptide), other organic molecule, inorganic molecule, or combination thereof, that has one or more effects on a biological system.

Proteomics: Global, whole-cell analysis of gene expression at the protein level, yielding a protein profile for a given cell or tissue. The comparison of two protein profiles (proteomes) from cells that have been differently treated provides information on the effects the treatment or condition has on protein expression and modification. Subproteomics is analysis of the protein profile of a portion a cell, for instance of an organelle or a protein complex. Thus, a mitochondrial proteome is the profile of the protein expression content of a mitochondrion under certain conditions.

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate). Likewise, a purified organelle preparation is one in which the specified organelle is more pure than in its natural environment within a cell, so that only relatively insubstantial amounts (e.g., less than 10% relative) of other organelles (or markers for other organelles) are present in the preparation.

Separate(d)/Separation: To spatially dissociate components, such as biomolecules. The components (for example, proteins or peptides) are usually separated based on one or more specific characteristics, such as molecular weight or mass, charge or isoelectric point, conformation, association in a complex, and so forth. Separation may be accomplished by any number of techniques, such as sucrose gradient centrifugation, aqueous or organic partitioning (e.g., 2-phase partitioning), non-denaturing gel electrophoresis, isoelectric focusing gel electrophoresis, capillary electrophoresis, isotachyphoresis, mass spectroscopy, chromatography (e.g., HPLC), polyacrylamide gel electrophoresis (PAGE, such as SDS-PAGE), and so forth.

Once a sample is subjected to a separation, it can be divided into sub-samples or fractions. These fractions may be divided in an order, which may be correlated for instance with a characteristic that was used to separate the components. Thus, a sample subjected to sucrose gradient separation can logically be divided into fractions based on the final density. Proteins or other biomolecules that are separated by an isoelectric focusing gel can be fractionated (e.g., the gel divided into strips) that are correlated with their net charge. Likewise, molecules subjected to SDS-PAGE separation can be fractionated based on their molecular weight. The division of a separated sample into fractions, in some order based on that separation, is well known to those of ordinary skill in the art.

As used herein, separation is not an absolute term (in that separation need not be perfect or “complete” for components to be “separated”). Thus, when a sample is subjected to a separation technique and the resultant separated sample is divided into fractions (e.g., fractions from a sucrose gradients, bands from a gel, and so forth), components within the sample can still be referred to as “separated” even though they occur in more than one of the fractions.

Subject: Living multi-cellular vertebrate organisms, a category that includes both human and non-human mammals.

Unless otherwise explained, all technical and scientific terms used herein have the same meaning as commonly understood by one of ordinary skill in the art to which the invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

III. Overview of Several Embodiments

A first embodiment is a method for creating a three-dimensional representation of a protein complement of a biological sample (e.g., a sample from an animal, a plant, a microbe, a fungus, or so forth). Examples of this method involve separating proteins contained in the biological sample using a non-denaturing separation process to produce a separated sample; dividing the separated sample into a plurality of identifiable sub-fractions having an order (for instance, the order in which they are removed from the separation); subjecting at least two of the sub-fractions to at least one denaturing separation process (though the sub-fractions need not be subjected to the same process) based on protein size and at least one separation process based on protein charge (though the sub-fractions need not be subjected to the same process), to produce a two-dimensional separation of the proteins in the sub-fractions; producing a representation of each two-dimensional separation of proteins; and assembling (e.g., through or involving computer processing) the plurality of representations in order to produce a three-dimensional representation of the proteins in the sample.

In specific embodiments, the non-denaturing process comprises separation on an osmotic gradient, for instance a discontinuous or continuous gradient, such as a sucrose gradient.

In specific embodiments, the denaturing separation process based on protein size comprises separation on a SDS-PAGE gel.

In certain embodiments, the denaturing separation process based on protein charge comprises separation on an isoelectric focusing gel.

It is particularly expected that in some embodiment, the denaturing separation process based on protein charge is carried out before the denaturing separation process based on protein size.

Also provided are methods for creating a three-dimensional representation of a protein complement of a biological sample, where the biological sample is an organellar preparation. In particular examples of such methods, the organellar preparation is a preparation enriched (e.g., by 20%, by 40%, by 60%, by 80%, or more compared to a starting sample) for nuclei, endoplasmic reticulum, mitochondria, plastids, lysosomes (vacuoles), peroxisomes, cytosol components, or plasma membranes. Specific envisioned methods are methods wherein the organellar preparation is a preparation enriched for mitochondria.

In further methods, the biological sample is prefractionated into a plurality of pre-fractions prior to being separated into sub-fractions using a non-denaturing separation process, and the three-dimensional representation of the proteins is a three-dimensional representation of a pre-fraction. In examples of such methods, the method of claim 13, where assembling the plurality of representations in order to produce a three-dimensional representation of the proteins in the sample further comprises assembling a plurality of three-dimensional representations of individual pre-fractions into a single three-dimensional representation.

Particular embodiments provide methods for creating a three-dimensional representation of a protein complement of a biological sample, which methods involve subjecting each of the sub-fractions to at least one denaturing separation process based on protein size (though not all sub-fractions need be subjected to the same process) and at least one separation process based on protein charge (though not all sub-fractions need be subjected to the same process), to produce a two-dimensional separation of the proteins in the sub-fractions.

The disclosure also provides three-dimensional representations of the protein complement of a sample, which representations are generated by any one of the disclosed methods.

A further embodiment includes a method for creating a three-dimensional representation of a protein complement of a mitochondrial sample, which method involves separating proteins contained in the mitochondrial sample using a sucrose gradient to produce a separated sample; dividing the separated sample into a plurality of identifiable sub-fractions having an order (such as the order in which the samples are removed from the sucrose gradient); subjecting at least some of the sub-fractions to isoelectric gel electrophoresis, followed by SDS-PAGE, to produce a two-dimensional separation of the proteins in the sub-fractions; producing a representation of each two-dimensional separation of proteins having a plurality of individual features; and assembling the plurality of representations in order to produce a three-dimensional representation of the proteins in the sample. Examples of such methods further involve identifying at least one feature in the three dimensional representation.

Methods described herein are methods of generating a three-dimensional protein profiles of biological samples. In particular embodiments, such methods are methods of generating a three-dimensional protein profile for a disease or condition, wherein the biological sample is a sample from an organism known to be afflicted with the disease or condition. An example of such an embodiment method is a method where the disease or condition is linked to mitochondrial function, the three-dimensional protein profile generated is a mitochondrial disease/condition-linked profile, and the biological sample comprises an organellar preparation enriched for mitochondria.

A further embodiment is a method of screening for a compound useful in treating, reducing, or preventing a disease or condition linked to mitochondrial function, or development or progression of a disease or condition linked to mitochondrial function, which method involves determining if application of a test compound to a subject alters a mitochondrial disease/condition-linked profile produced from the subject, so that the profile less closely resembles a mitochondrial disease/condition-linked profile than it did prior to such treatment, and selecting a compound that so alters the profile.

Yet another embodiment is a method of determining drug or treatment effectiveness or side effects, which method involves applying a drug or treatment to an organism or a cell sample from the organism; taking a biological sample from the organism or the cell sample from the organism; analyzing the biological sample to produce a test three dimensional protein profile for the subject; comparing the test three dimensional protein profile for the organism with a control three dimensional protein profile (for instance, a profile generated prior to the treatment or drug application); and drawing conclusions about the effectiveness or side effects of the drug or treatment based on differences or similarities between the test three dimensional protein profile and the control three dimensional protein profile. In specific examples of such methods, the drug or treatment is a drug or treatment for a mitochondrial-linked disease or condition, and the test and control three dimensional protein profiles are three dimensional mitochondrial protein profiles.

IV. Enhanced Protein Analysis

The methods described herein provide enhanced protein separation techniques, which facilitate proteomic analysis of living systems. In one embodiment, sucrose gradient centrifugation is combined with two-dimensional gel electrophoresis to produce a three-dimensional representation of the proteome. The resulting three-dimensional separation of proteins addresses several of the problems encountered during previous attempts to obtain complete proteome maps, such as resolution of proteins and solubility of hydrophobic proteins during isoelectric focusing. In addition, the new protein separation techniques described herein provide functional information about protein complexes within an organelle (or other subcellular division) that is not obtained with two-dimensional gel electrophoresis of, for instance, whole mitochondria or a whole cell.

As mitochondria play critical roles in both cell life and cell death, there is great interest in obtaining a human mitochondrial proteonie map. Such a map is expected to be useful in diagnosing diseases, identifying targets for drug therapy, and in screening for drug effects and side effects. The mitochondrion was therefore used as an example system to explore the potential of the described protein separation techniques. Other protein systems can be analyzed with the methods provided herein in, including for instance other organelles, as well as specific tissue or cell types. The methods herein are therefore not intended to be limited to analysis of mitochondrial protein analysis.

In a representative example described in detail herein, mitochondrial proteins are separated based on their associations in the organelle (as shown in FIG. 6). A significant number of mitochondrial proteins exist in vivo as multi-polypeptide complexes. Examples include the five complexes of the oxidative phosphorylation machinery (Saraste, Science, 283:1488-1493, 1999), the mitochondrial ribosome (Curgy, Biol. Cell, 54:1-38, 1985), the mitochondrial nucleoid (a complex of mtDNA and assorted nucleotide binding proteins; Newman et al., Nucleic Acids Res., 24:386-393, 1996), as well as the TIM and TOM complexes (Pfanner and Meijer, Curr. Biol., 7:R100-R103, 1997) involved in protein import into the organelle, and the permeability transition pore, which has been linked to apoptosis (Fontaine and Bernardi, J. Bioenerg. Biomembr., 31:335-345, 1999).

The first step of this embodiment of the described separation analysis is a discontinuous sucrose gradient that separates the component polypeptides by the sizes of the complexes in which they participate. The effectiveness of this separation method is demonstrated herein using the OXPHOS component proteins, whose location could be readily followed using this laboratory's set of monoclonal antibodies specific to these complexes. During the course of this study, the protein patterns reported here have been obtained more than 50 times, confirming that this method is highly reproducible. In addition, a similar separation of complexes is obtained for various tissue samples, demonstrating the broad applicability of the described separation techniques.

An advantage of the sucrose gradient pre-fractionation step (or other primary separation) for proteome analysis is that it separates the total protein complement of the starting sample into “workable” fractions for subsequent electrophoretic separation, as well as providing functionally relevant information (i.e. assembly state, activity) about the various proteins. This simplification afforded by separating total proteins into fractions allows some of the problems encountered in previous proteome attempts to be dealt with in a manageable manner. With fewer proteins per sample, the same fraction can be run on multiple gels of varying isoelectric point ranges, which helps solve the “range of isoelectric point” problem while still producing simple enough patterns for subsequent analysis.

In addition, during conventional 2-D electrophoresis, many hydrophobic proteins are lost during the initial isoelectric focusing step. Information about the hydrophobic proteins, which would have normally been lost during the isoelectric focusing step, now can be obtained by identifying the fragment sizes in the mass spectrometry analysis which are present in the one-dimensional gel but are absent in the 2-D-gel. With the herein-described separation methods, the same sample can be subjected both to one-dimensional SDS-PAGE and to 2-D gel electrophoresis (consisting of isoelectric focusing followed by SDS-PAGE). There are fewer proteins in each of the fractions produced using sucrose-gradient pre-fractionation than in whole mitochondrial samples. Recent advances in mass spectrometry allow for identification of individual components in mixtures of proteins, and therefore mass spectrometry can be carried out on both the individual spots in the described 2-D-gel and on the equivalent size band from the one-dimensional gel.

Two other useful aspects of the described prefractionation are that activity measurements can be obtained from the same fractions being subjected to electrophoresis and that proteins present in low copy number can be concentrated into one of the fractions. Activity measurements provide the added information of whether the complexes being studied are functional (and/or to what extent they are functional). By concentrating low copy number proteins into smaller fractions, detection is made easier. By way of example, results are discussed below that demonstrate that the subunits of Complex I are concentrated in fraction 1. Samples produced using methods described herein can and are being examined to detect proteins of even lower abundance such as SURF-1, a protein that catalyzes cytochrome c oxidase assembly.

A. Types of Proteomes

The protein separation methods described herein can be used to provided enhanced separation and identification to any protein system, and are not limited to the example systems presented in detail herein. In essence, any proteome can be generated using the described techniques; the larger the number of component proteins, the more advantageous it is to pre-fractionate the protein sample prior to electrophoretic analysis as described herein. Thus, the described methods can be used to generate proteomes from various organisms, including microbes, plants, animals (for instance, humans).

The described enhanced protein separation methods can also be used to produce proteomes for sub-cellular fractions to produce subproteonies (e.g., on an organelle by organelle basis, or system by system within a cell). Sub-proteomes can be produced from any cell fraction that can be reliably produced. Representative examples of sub-proteomes that can be analyzed using the described enhanced protein separation methods include (but are not intended to be limited to): nuclear, mitochondrial, lysosomal/vacuolar, endoplasmic reticulum ER), secretory system as a whole, plastid (e.g., chloroplast), peroxisomal, and cytosol (not all of which will be found in all cells).

Proteomes can be assembled for whole cells using the described techniques; the proteins from whole cells are advantageously sub-divided (for instance, by organelle) prior to non-denaturing (e.g., sucrose gradient) separation and subsequent electrophoretic analysis. Thus, individual sub-cellular proteomes such as those described above can be assembled (for instance, using a computer system) into the comprehensive proteome of a whole cell. However, such sub-division of the cell is not essential. Entire cell protein preparations can be separated on long sucrose gradients, and numerous fractions collected for subsequent denaturing analysis. It is, however, advantageous in some embodiments to take advantage of the compartmentalization of eukaryotic cells to further simplify the protein profile being examined.

The described techniques also permit enhanced detection of protein-interaction perturbations caused by protein co- and/or post-translational modification. Since such modifications often influence and/or control the ability of proteins to interact in complexes, the non-denaturing separation that is integral to the described protein separation methods permits separation of differentially modified protein forms. This is believed to simplify the interpretation of proteomic data, as well as providing more information on the functional forms of specific proteins. Co- and post-translational modifications are discussed, for instance, in Chapter 4 of Wilkins et at., (Proteome Research: New Frontiers in Funational Genomics, Springer-Verlag, Berlin, 1997; ISBN 3-540-62753-7).

The pivotal importance of the mitochondrion makes it an excellent example protein system in which to demonstrate the effectiveness and reliability of the described enhanced protein separation techniques. The mitochondrion is important in several cellular processes, including the generation of “energy” and programmed cell death (apoptosis). In addition, defects in this organelle contribute to, and are frequently a primary cause of, many human diseases. These defects are often caused by mutations in mitochondrial proteins such as enzymes involved in fatty acid metabolism and oxidative phosphorylation (Eaton et al., Biochem. J., 320:345-357, 1996; Wallace, Science, 283:1482-1488, 1999). Freidrich's Ataxia is just one example of a disorder that is known to be caused by a mutated mitochondrial protein (Lodi et al., Proc. Natl. Acad. Sci. USA, 96:11492-11495, 1999). Other diseases linked to defective mitochondrial proteins are being reported with increased frequency (Scharfe et al., Nucleic Acids Res., 28:155-158, 2000). Mitochondria also play a key role in apoptosis or programmed cell death (Mignotte et al., Eur. J. Biochem., 252:1-15, 1998). Enhanced rates of cell death, due in part to mitochondrial dysfunction, are now considered to be an important component of Alzheimer's, Parkinson's, and Huntington's diseases (Lemasters et al., J. Bioenerg. Biomembr., 31:305-319, 1999; Beal, Trends Neurosci., 23:298-304, 2000; Schapira, Biochim. Biophys. Aca, 1410:159-170, 1999). Mitochondrial dysfunction can also lead to decreased rates of cell death, and roles for mitochondria in cancer have been described (Polyak et al., Nat. Genet., 20:291-293, 1998; Fliss et al., Science, 287:2017-2019,2000). In addition, many drugs used in treatment of diseases such as cancer and AIDS have mitotoxic effects. For instance, AZT can be problematic for patients due to severe disruptive mitochondrial effects (Yerroum et al., Acta Neuropathol., 100:82-86, 2000).

B. Separation Techniques

Certain enhancements arising from the separations described herein are accomplished by the combination of a non-denaturing pre-separation of a protein sample into less-complex sub-samples (fractions), followed by subsequent denaturing separation. Various specific separation techniques can be used for each of these two portions of the separation, and indeed it is contemplated that different separation techniques can be employed to separate different sub-fractions from the same original biological sample. In general, however, the first separation technique employed is one that retains or substantially retains the functionality of at least one complex of interest in the protein sample.

One example of the first separation is a size or buoyant density gradient separation method, such as a discontinuous sucrose gradient, that separates the component polypeptides of the sample by the sizes of the complex(es) in which they participate. Sucrose gradients for the separation of proteins are well known, and modifications to the disclosed sucrose gradient method are contemplated. Such modifications may include the use of a continuous rather than discontinuous gradient and different gradient conditions (for instance, different sucrose concentrations, different buffers, or different osmoticum). The length of the gradient can also be varied, with longer gradients expected to give better overall separation of proteins and protein complexes, and to provide a larger number of fractions that are then each individually analyzed using a denaturing system.

Other “mild” separation techniques that are suitable for the first separation phase include aqueous 2-phase partitioning and non-denaturing agarose gel electrophoresis separation (such as native blue gels):

Once the original protein sample is pre-fractionated into a few to several fractions, one or more usually two additional separations are performed; the order of these subsequent separation phases is not critical, but for ease of description they will be referred to as the second and third separation phases.

In specific embodiments, each of the fractions (or a select subset of them, for instance a cluster of fractions, every other fraction, every third, and so forth) produced by the first separation is further separated using net charge and size, usually in a denaturing system. For instance, in the second separation phase of the procedure, the individual proteins in a complex are separated by net charge. Typically, this occurs by separation in an isoelectric focusing (IEF) gel. Other techniques for separating and isolating the proteins include capillary electrophoresis or isotachyphoresis. In many instances, non-protein components in the sample are removed during preparation of the sample(s) for IEF.

In the third separation, the individual proteins are separated by size (e.g., by SDS-PAGE or sizing gel, or by mass spectroscopy). Mass spectroscopy may be performed after separated proteins are fragmented with an enzyme (such as trypsin) or a chemical cleaving agent (such as cyanogen bromide). The peptide mass profile (peptide fingerprint) obtained from mass spectrometry is compared with theoretical fragmentation patterns derived from sequence date in genomic databases in order to aid in identifying the proteins. Additionally, Edman sequencing can be used in identifying peptides.

Representative examples of such separation techniques are presented below, in Examples 3 and 4; representative results from an analysis of the mitochondrial proteome are presented in the accompanying figures. Other examples of two-dimensional electrophoretic analysis are well known; see, for instance, Chapter 2 of Wilkins et al. (Proteome Research: New Frontiers in Functional Genomics, Springer-Verlag, Berlin, 1997; ISBN 3-540-62753-7).

Proteins can be visualized on denaturing gels using any of various known stains. However, some stains are more advantageous than are others. For instance, the use of SyproRuby™ dye (Molecular Probes, Oregon) allows seamless throughput from the gels to mass spectrometry, as well as providing the best sensitivity available to date in staining individual proteins for identification.

Traditional buffering systems can be used for separating proteins in the component fractionations of the described systems. However, as is well known to those of ordinary skill in the art of protein separation, minor modifications to such buffer conditions can be made to optimize the buffers for individual raw protein preparations (see, for instance, the discussion of two-dimensional electrophoresis in Chapter 2 of Wilkins et al., Proteome Research: New Frontiers in Functional Genomics, Springer-Verlag, Berlin, 1997; ISBN 3-540-62753-7). Possible modifications include, for instances, changes in the pH, osmoticum (e.g., sucrose), salt content and/or concentration of individual solutions (e.g., the solutions used to make gradients, gels, and/or the buffers used to run the gels). The temperature, voltage, and amperage at which individual gels are run also can be modified, as can the speed and duration of gradient equilibration and centrifugation. One of ordinary skill in the relevant art will know not only how to vary these and other relevant conditions, but will also know the effects such variations are likely to have on the operation of the system (e.g., the likely effects on protein separation). All such minor variations of conditions that are used to optimize separation conditions are encompassed herein.

C. Identification of Individual Features

Proteins separated using the herein-described enhanced techniques can be analyzed using any of various well-known techniques. For instance, those protein identification techniques currently used to analyze individual protein features in 2-D proteomes can be used. Such techniques are well known, and examples can be found for instance in Wilkins et al. (Proteome Research: New Frontiers in Functional Genomics, Springer-Verlag, Berlin, 1997; ISBN 3-540-62753-7). In particular, Chapter 3 of Wilkins et al. provides insights on such techniques. Examples of applicable protein identification techniques include (but are not intended to be limited to) protein activity assays (see Example 5); antibody recognition (Western mapping using, for instance, mAbs to individual known proteins; see Example 6); direct comparison to previous proteomic maps (on which features have been identified through any method); mass spectrophotometry; and database screening for peptide sequence matches, for instance using peptide(s) removed from a gel or blot.

D. Raw Data, Data Assembly, Automation, and Data Analysis

The form of data presentation from the described enhanced protein separation methods is largely a matter of individual preference. For instance, the individual gels produced can be viewed individually, as is discussed below in specific examples. Though the raw data resulting from the sequential protein separations can be read by an individual, it is advantageous considering the vast amount of information contained in each protein profile to process the data using a computer.

In certain embodiments, therefore, it is advantageous to scan stained gels and/or blots produced using the herein-described separation methods into a computer for the processing and/or analysis of the raw data. Programs exist, and more are being developed, that permit subtraction of gel or stain artifacts, calculation of relative and/or apparent pI, molecular weight, and amount of each protein feature, and/or calculation of protein-protein interactions (for instance by comparing different pre-fractionated samples produced using the described methods).

The computer-assisted comparison of multiple gels can be used to detect changes in proteomes, which changes can be linked to (for instance) disease progression, environmental or other stimuli, clinical treatment, developmental changes, and so forth. Such comparisons also permit standardization of gel results, for instance by consistently identifying features between different gels (such as gels produced in different laboratories, or using proteins from different samples).

Computer scanning of the described protein gel profiles also permits the assembly of a set (or sub-set) of pre-fractionated samples into a three-dimensional map, such as is displayed in the simplified model shown in FIG. 6. In this example, individual gels (the Z dimension) represent different fractions from a non-denaturing discontinuous sucrose gradient; each fraction has subsequently been further separated using isoelectric focusing (the X dimension) and denaturing SDS-PAGE analysis (the Y dimension).

V. Applications

With the provision herein of systems for enhanced protein separation and the generation of fine-detail, three-dimensional representations of protein profiles of tissues, cells, organelles and so forth, the use of these representations to accurately compare protein profiles under different conditions is enabled. This excellent comparison system can be used, for instance: (1) to identify novel or previously unidentified proteins (for instance in an organelle, such as the mitochondrion); (2) in detection and diagnosis of disease, disease state, or prediction of disease progression (prognosis); (3) in development and testing of pharmaceutical agents; (4) for tracking of drug efficacy in a subject, and (5) for tracking of drug toxicity in a subject. Sample comparison can be between healthy and diseased tissues (e.g., biopsy) or cells (or cell cultures), diseased tissue at different stages (e.g., different cancer stages or the stages of other progressive diseases), tissue before and after drug or other treatment, and so forth.

Some clinical, biomedical, and biological applications of proteomics are described, for instance, in Chapters 8 and 9 of Wilkins et al. (Proteome Research: New Frontiers in Functional Genomics, Springer-Verlag, Berlin, 1997; ISBN 3-540-62753-7).

Specific embodiments are illustrated by the following non-limiting Examples.

### EXAMPLES

General Materials and Methods

Materials used for biochemistry were from Sigma Chemical Company (St. Louis, Mo.), unless otherwise stated. Laurylmaltoside (LM) was purchased from Calbiochem (La Jolla, Calif.). IPG strips 3-10 (18 cm) were purchased from Amersham Pharmacia Biotech (Piscataway, N.J.). SyproRuby™ protein gel stain was obtained from Molecular Probes Inc. (Eugene, Oreg.). All chemicals used for 2-D electrophoresis were from Genomic Solutions (Ann Arbor, Mich.).

Trichloroacetic acid (TCA) precipitation was done according to Petersen (Anal. Biochem., 83:346-356, 1977). One-dimensional mini gels were run essentially according to Laemmli (Nature, 227:680-685, 1970) using 10-20% gradient polyacrylamide gels. The gels were stained with Coomassie Brilliant Blue (Downer et al., Biochemistry, 15:2930-2936, 1976) or SyproRuby™ protein gel stain using known procedures (Berggren et al., Electrophoresis, 21:2509-2521, 2000).

### Example 1

### Preparation of a Biological Sample

This example provides descriptions of how one sample type, isolated mitochondria, can be prepared from various tissues for analysis using the separation systems described herein. Other tissue, cell, or subcellular preparations also can be examined; such samples can be prepared using any conventional means.

In certain embodiments, it is beneficial that the final preparation is not substantially denatured (e.g., so that in vivo protein-protein interactions have been substantially maintained). In general, the more pure the target sample is, the better the results will be from the proteomic analysis.

Preparation of Mitochondria from Bovine Heart

All steps for purifying mitochondria were done at 4° C. unless otherwise stated.

The ventricles of a fresh bovine heart were cleaned of any connective tissue and fat before being minced into small pieces. About 600 ml of a Tris/sucrose buffer (0.2 mM EDTA, 0.25 M sucrose, 10 mM Tris/HCl pH 7.8) was added to 300 g of minced tissue and then blended in a Waring Blender for 30 seconds at high speed followed by 30 seconds at low speed. The pH was checked and, if necessary, adjusted to 7.8 with 2 M Tris before repeating the blending and adjustment of pH. The blended tissue was homogenized further with an Ultraturrex (Kinematica, Switzerland) (3.5 seconds at speed 9) followed by additional pH adjustment if needed. The homogenate was centrifuged at 185×g for 15 minutes in a KAJ-9 (Becknan, USA) rotor and the supernatant was filtered through four layers of cheesecloth. The filtrate was homogenized in a glass homogenizer with a tight fitting Teflon pestle and centrifuged at 740×g for 10 minutes in a KAJ-9 rotor. The pellet was discarded and the resulting supernatant was centrifuged at 20,600×g for 15 minutes in a GSA rotor to pellet the mitochondria. The pellets were washed twice in the Tris/sucrose buffer supplemented with 0.5 mM PMSF before the final pellet was resuspended in a small amount of buffer. After determining the protein concentration the mitochondria were frozen at −80° C.

Preparation of Mitochondria from MRC-5 Fibroblasts

MRC-5 fibroblasts were obtained from the American Type Culture Collection. The population doubling (PD) of the cells was in the range of 35-45 before harvesting to isolate mitochondria. Rho⁰-MRC5 fibroblasts were derived by culturing MRC-5 fibroblasts (PD=28-30) continuously in media supplemented with 50 ng/ml ethidium bromide for a further 16 PD's. All cells were grown as described before (Marusich el al., Biochim. Biophys. Acta, 1362:145-59, 1997) in high glucose Dulbecco's modified Eagle's medium, supplemented with 10% bovine calf serum, 50 μg/ml uridine, 110 μg/ml pyruvate, and 10 mM HEPES buffer to maximize growth rates.

For the preparation of mitochondria, 12-16 plates (150 mM diameter) of confluent MRC-5 fibroblasts were harvested, and the cells were washed three times in Ca²⁺, Mg²⁺ free phosphate buffered saline (CMF-PBS). To improve the cell disruption, the cell pellets were frozen at −80° C. for at least an hour. After thawing, 5 ml of homogenization buffer (0.5 μg/ml leupeptin, 0.5 μg/ml pepstatin, 1 mM PMSF, 250 mM sucrose, 1 mM EGTA, 1 mM EDTA, 10 mM HEPES/NaOH pH 7.4) was added and the pellets were homogenized in a glass homogenizer with a Teflon pestle. The homogenate was centrifuged (1,500×g, 10 minutes, 4° C.) and the supernatant was transferred into a clean tube. The homogenization was repeated twice with the pellet, and the supernatants were combined. The three combined supernatants were centrifuged (1,500×g, 10 minutes, 4° C.) and the pellet discarded. The resulting supernatant was once more centrifuged to pellet the mitochondria (10,000×g, 12 minutes, 4° C.). The supernatant was discarded and the pellet was resuspended in 5 ml wash buffer (0.5 μg/ml leupeptin, 0.5 μg/ml pepstatin, 1 mM PMSF, 250 mM sucrose, 1 mM EGTA, 1 mM EDTA, 10 mM Tris/HCl pH 7.5). The centrifugation was repeated (10,000×g, 12 minutes, 4° C.) and the final pellet was resuspended in 200-500 μl wash buffer. After measuring the protein concentration, the mitochondria were frozen at −80° C.

Preparation of Mitochondria from Human Brain Tissue

Human brain tissue was obtained from the Harvard Brain Tissue Resource Center, which is supported in part by PHS grant number MH/NS 31862. The mitochondria from human brain tissue were essentially prepared as described for MRC-5 fibroblasts and were a kind gift of Dr. Leslie A. Shinobu (Massachusetts General Hospital).

Further modifications in the described methodologies may improve the data that can be produced using the described system for protein separation and proteome analysis. One such modification would be to improve the purity of the mitochondrial preparation used. Such modification likely may be limited by recent evidence of interaction between the mitochondrion and other organelles, e.g., the ER (Rizzuto et al., Science, 280:1763-1766, 1998).

### Example 2

### Non-denaturing Separation of the Biological Sample

This is a representative example of a non-denaturing separation technique, discontinuous sucrose gradient analysis, which can be used to separate biological components based on their protein-protein interactions.

Separation of Mitochondrial Proteins by Sucrose Gradient Fractionation

Two slightly different sucrose gradients have been employed for the separation of mitochondrial complexes after extraction. The first gradient is optimized for the purification of the respiratory chain complex I, whereas the second is optimized for the use in 2-DE (two dimensional electrophoresis). These gradients are referred to herein as gradient A and gradient B, respectively.

Mitochondria prepared from three different sources (bovine heart, cultured MRC-5 fibroblasts, and human brain) as described above, were solubilized for analysis using 1% LM. Mitochondria (1-5 mg) were pelleted (TLA 100.2 Beckman rotor, 10,000×g, 10 minutes, 4° C.) and resuspended at a protein concentration of 5 mg/ml in 100 mM Tris/HCl, 1 mM EDTA, pH 7.5, 1 μg/ml pepstatin, 1 μg/ml leupeptin, 1 mM PMSF, 1% LM. The mitochondria were incubated in this solution for 20 minutes on ice with stirring, before the membranes were pelleted again by centrifugation (TLA100.2, 185,000×g, 20 minutes, 4° C.). The supernatant (250 μl, 500 μl, or 1 ml) was layered on top of a sucrose gradient. Composition of gradient A: 250 μl (35%), 500 μl (30%), 750 μl (27.5%), 1 ml (25%), 1 ml (20%), 1 ml (15%). Gradient B: 500 μl each of the following sucrose concentrations: 35%, 32.5%, 30%, 27.5%, 25%, 22.5%, 20%, 17.5%, 15%. The 35% fraction was omitted, when 1 ml of supernatant was to be applied to the gradient. Both gradients were centrifuged overnight at 4° C. (150,000×g, 16.5 hours, SW 50.1, acc. 7, dec.7). All sucrose solutions contained 100 mM Tris/HCl, 0.05% LM, 1 mM EDTA. The sucrose gradient was fractionated into nine fractions from the bottom of the tube into 500 μl fractions, which were frozen at −80° C.

### Example 3

### Denaturing Separation of the Biological Sample

This example provides one method for further separating proteins in fractions of a sucrose gradient, using denaturing gel electrophoresis, specifically SDS-PAGE. In some embodiments, this separation step is performed immediately after separation of the sample using a non-denaturing system (e.g., sucrose gradient fractionation). In other embodiments, fractionated samples are first subjected to isoelectric focusing gel analysis, then applied to a denaturing gel for final analysis.

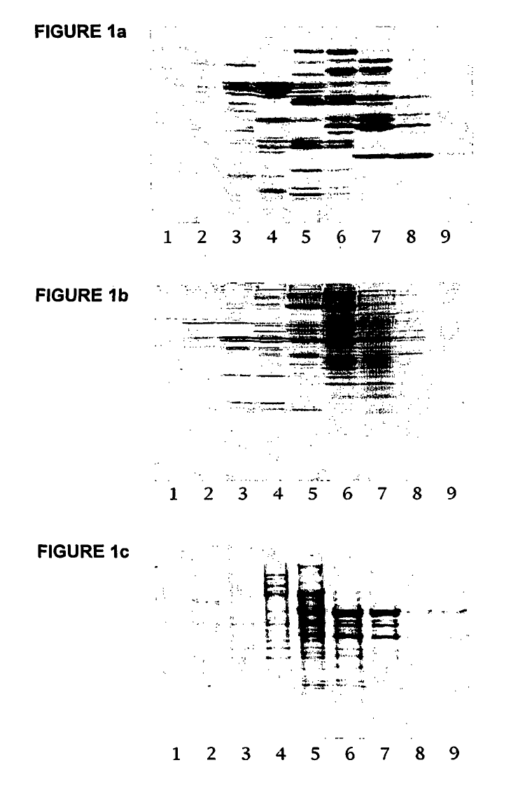

For SDS-PAGE analysis, 10-20 μl of each fraction was loaded per lane. The composition of fractions after SDS-PAGE and subsequent staining with SyproRuby™ protein gel stain is shown in FIG. 1.

Results

There is considerable difference in the overall staining pattern between the three different tissue samples, but this is to be expected for several reasons. First, heart tissue is rich in mitochondria and the mitochondria are easily purified essentially free of other organellar membranes. This is not the case for brain or cultured fibroblasts. Brain and fibroblast mitochondria isolated by differential centrifugation can contain many other vesicular membranes that are closely connected to the mitochondria, including endoplasmic reticulum (ER) and the Golgi apparatus. Indeed, small amounts of both ER and Golgi proteins have been shown to be present in our purified brain and fibroblast mitochondrial samples as indicated by Western blot analysis using organelle specific antibodies. Furthermore, based on quantitative Western blotting with mAbs to each complex, the levels of respiratory chain proteins per mg total mitochondria protein is three times higher in heart than in brain and 6-7 times higher in heart than in cultured fibroblasts.

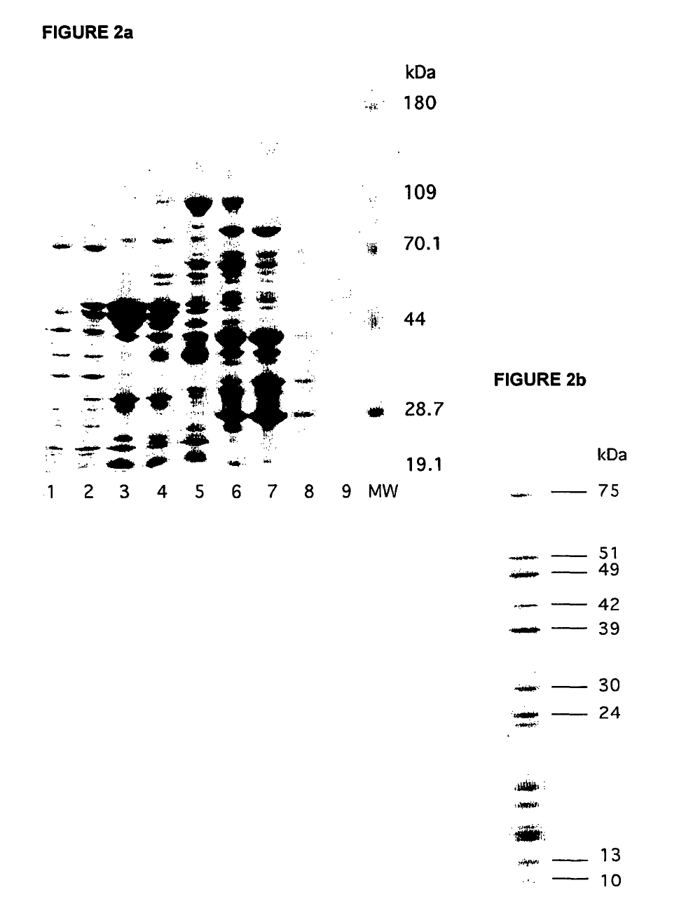

The experiments shown in FIG. 1 use only a portion of each sucrose gradient fraction, leaving sufficient material for enzyme assays and additional gel electrophoresis analysis. However, to ensure better visualization of low copy number proteins, entire sucrose gradient fractions may be TCA precipitated and subjected to 10-20% gradient SDS-PAGE (FIGS. 2a,b). FIG. 2b and lane 1 of FIGS. 1 and 2a include proteins in complexes larger than 700,000 Da that are still assembled after 1% LM treatment. As shown in FIG. 2b, the gel pattern obtained from the first fraction of Gradient A matches previously published patterns for complex I (Walker et al., Methods Enzymol., 260:14-34, 1995) for the molecular weight range analyzed. This complex contains at least 42 different polypeptides (Skehel et al., FEBS Lett., 438:301-305, 1998), approximately half of which are larger than 19 kDa, and quantitative estimates of the levels of complex I in beef heart mitochondria range from 60 to 130 pmol/mg mitochondrial protein (Smith et al., FEBS Lett., 110:297-282, 1980; Albracht et al., FEBS Lett., 104:179-200, 1979). Thus, complex I is enriched in fraction 1 after the gradient and can be easily visualized after TCA precipitation.

### Example 4

### Second Denaturing Separation of the Biological Sample

This example provides one method for further separating proteins in fractions of a sucrose gradient, using isoelectric focusing gel electrophoresis.

For the IEF dimension, 50-100 μl sample as prepared in Examples 1 and 2 was used for each strip. To each sample, 3 μl 10% SDS and 12.5 μl 10% LM were added before adjusting the total volume to 500 μl with urea sample buffer (5 M urea, 2 M thiourea, 2% CHAPS, 1% zwittergent 3-10, 0.8% carrier ampholytes, 65 mM DTT). Each IPG (immobilized pH gradient) strip was rehydrated in this solution overnight. The IEF dimension was run in the pHaser isoelectric focusing unit (Genomic Solutions) as suggested by the manufacturer.

After reaching equilibrium, each strip was incubated in 2 ml of 375 mM Tris, 50 mM DTT, 3% SDS pH 8.6 for 10 minutes at room temperature with gentle shaking before being transferred to the second dimension.

For the second dimension, 10% polyacrylamide gels with a pH of 9.2 or 17.5% standard homogenous slab gels, both with 4.5% stacking gels, were used. The gels were run in the Investigator 2-D gel tank (Genomic Solutions) according to the manufacturer's protocol. After completion of the run, the gels were fixed in 10% methanol, 7% acetic acid for one hour and stained with SyproRuby™ protein gel stain as described in Berggren et al. (Electrophoresis, 21:2509-2521, 2000).

Imaging of the gels was carried out with an FLA3000 fluorescent image analyzer (Fuji Photo Film, Tokyo, Japan) with a 473 nm excitation filter and a 580 nm long pass emission filter.

Results

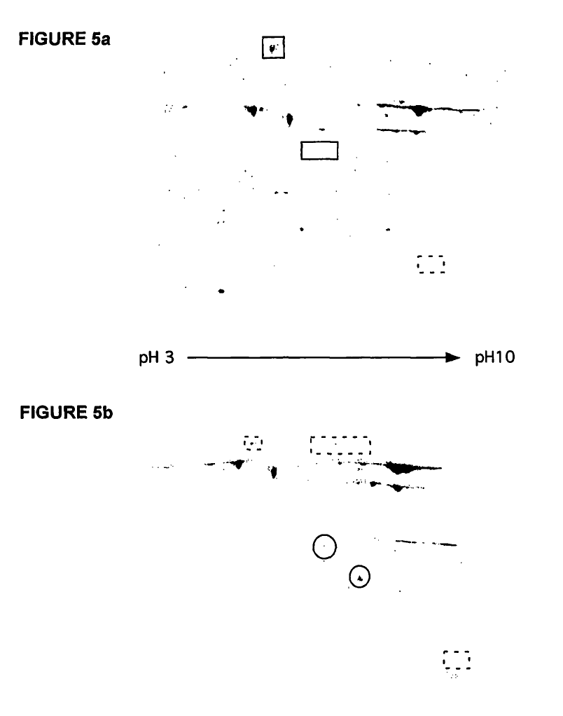

Representative results are shown in FIG. 5, which shows the profiles of two fractions of the sucrose gradient of bovine heart mitochondria. The protein profiles of the prefractionated mitochondria are greatly simplified compared to typical 2-D profiles of whole mitochondria. Fraction 3 contains 56 identifiable spots at the levels of protein loaded while fraction 4 contains about 90. In contrast, whole mitochondria preparations loaded at an equal protein amount show 350 clearly identifiable spots on a single gel. The number of spots on a single gel can be significantly increased by loading more protein; the resolution of proteins, however, decreases as the spot number increases. Using the disclosed 3-D methods, there is some overlap of polypeptide content between fractions, which may be seen as a complication in that the same protein is identified more than once. However, it is also an advantage in that patterns may be aligned using common “landmarks,” thus facilitating profile comparison and assembly of a three-dimensional visualization system. In FIG. 5, selected spots with very different intensities in the two fractions, or that are unique to an individual fraction, are circled or encased by squares.

Identification of the various spots can be carried out using mass spectrometry, for instance. Other approaches also can be used to identifying each spot, including complex-specific purification and immunologic (e.g., mAb based) identification.

### Example 5

### Identification of Individual Features in the Proteome: Activity Assays

This example provides illustrations of specific representative methods that can be used to identify individual proteins (features) within the multi-dimensional protein profiles described herein.

ATP Hydrolysis and Creatine Kinase Activity Measurements

To show that protein complexes are still functional after separation on the sucrose gradient, ATPase and creatine kinase activity measurements were carried out on each fraction of the sucrose gradient of bovine heart mitochondria. For the activity measurements, a 20 μl aliquot from each sucrose gradient fraction was used. ATP hydrolysis was measured with a regenerating system as described by Lötscher et al. (Biochemistry, 23:4140-4143, 1984). The creatine kinase activity was measured according to Bücher et al. (Handbuch der physiologisch-und pathalogisch-chemischen Analyse, Hoppe-Seyler/Thierfelder, vol. VI/A, Springer, Berlin, Göttingen, Heidelberg, 1964, pp. 292-339.).

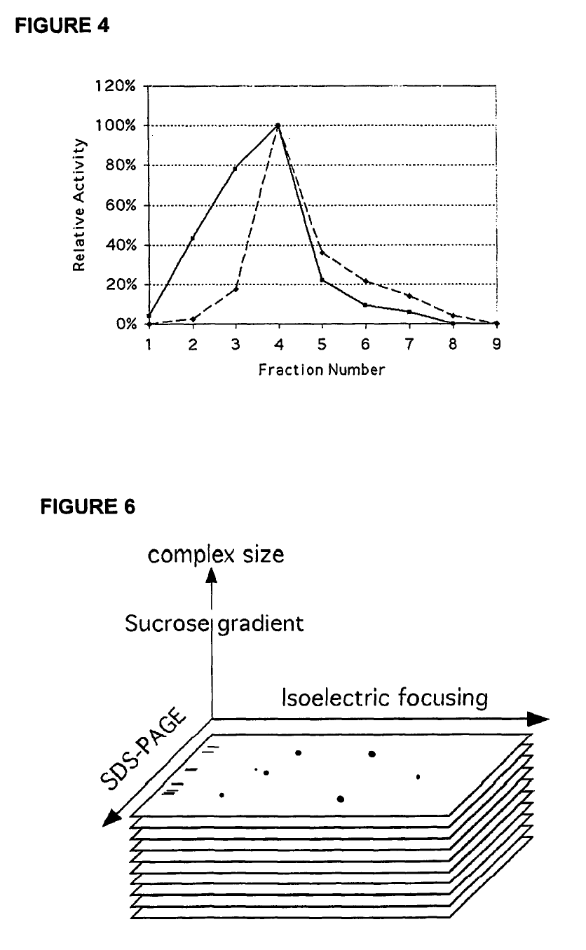

The activity in each fraction is expressed as a relative percentage of the maximum activity in the peak fraction (FIG. 4). The highest ATP hydrolysis activity was measured in fraction 4, which is in agreement with the position of complex V in the gradient, as indicated by the Western blot for complex V-α. Creatine kinase has been reported to form an octameric complex with an estimated molecular weight of 400 kDa (Schlegel et al., Biol. Chem., 263:16942-16953, 1988). Monomeric bovine heart complex V is estimated to be 550 kDa (Schagger et al., Anal. Biochem., 217:220-230, 1994). The creatine kinase activity peaks slightly after the ATP hydrolysis activity, in agreement with these estimated molecular weights.

### Example 6

### Identification of Individual Features in the Proteome: Western Blotting

In addition to the above-described methods, monoclonal antibodies can be used to identify individual proteins separated using the techniques described herein. At this time, mAbs specific to seven subunits of complex I, two of complex II, three of complex III, ten of complex IV, and three of complex V are available for such identification, for instance. This example provides descriptions of Western blotting using some of these mAbs to identify specific protein spots separated as described above.

Western Blotting of One Dimensional Gels

Western blotting was done essentially according to Marusich et al. (Biochim. Biophys. Acta, 1362:145-59, 1997) with the following modifications. Proteins were transferred to 0.45 μm polyvinylidine difluoride (PVDF) membrane (Millipore, Bedford, Mass.) using a semi-dry transfer system (Amersham Pharmacia Biotech) according to the manufacturer's specifications. Reactive bands were detected using the ECL Plus™ detection reagent (Amersham Pharmacia Biotech) and were imaged using the image analyzer Storm 860 (Molecular Dynamics, Sunnyvale, Calif.). Fluorescence was quantified using NIH Image. All antibodies used in this study were prepared in the monoclonal antibody facility at the University of Oregon. The antibodies were used at the following concentrations: Complex I 39 kDa (2 μg/ml), complex II 30 kDa (5 μg/ml), complex III Core 2 (0.4 μg/ml), complex IV COX II (2 μg/ml), complex IV COX Va (2 μg/ml), complex V alpha (4 μg/ml). The antibodies were all mouse monoclonals.

Results

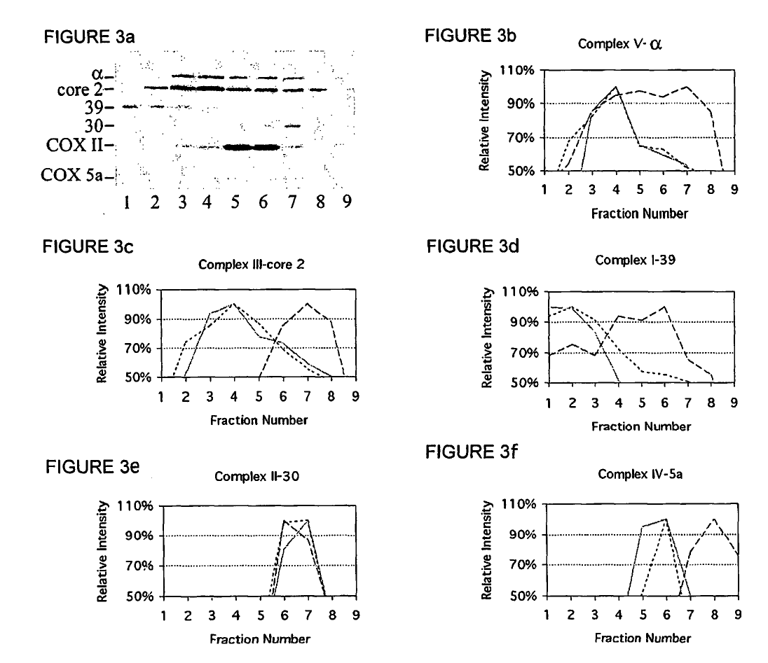

As shown in FIG. 3a, complex I runs the furthest in the gradient (based on the 39 kDa polypeptide), followed by complex V (α subunit) and complex III dimer (Core 2), complex IV (COX Va and II), and finally complex II (30 kDa subunit). This ordering of the complexes from highest molecular weight to lowest molecular weight is in accordance with previous estimates of their molecular weights (Schägger et al., Anal. Biochem., 217:220-230, 1994).

Densitometric scans of the Western blots from either MRC-5 fibroblasts or bovine heart proteins were quantified and, for convenience, the relative expression levels of each subunit in the various fractions were expressed as a percentage of the highest intensity band in the gradient. The distribution of each subunit in the gradient was then plotted (FIGS. 3b-f). The broad distribution seen for complex III likely arises in part because this complex can be a monomer as well as a dimer. The ATP synthase also broadly distributes; this is likely due to the partial disruption of the F₁F₀ into F₁ and F₀ components.

These plots are not representative of the absolute levels of the complexes, because the blots were developed to identify even small amounts of each complex. Therefore, the levels of protein present in the peak fractions are grossly under represented because of antibody saturation effects. A better measure of the levels of the complexes present in the fractions is the staining intensity of the bands in FIG. 1, which show that complexes III and V are concentrated in fraction 4. Nonetheless, the plots do reveal that the distribution of each complex in the sucrose gradient is nearly identical for the bovine heart and MRC-5 mitochondrial extracts.

In order to show that the sucrose gradient is sensitive to molecular weight changes and complex assembly, mitochondria from MRC-5 fibroblast lacking mitochondrial DNA (Rho⁰) were used. Though these fibroblasts are respiration-deficient, they can be cultured in a medium favoring glycolysis. Western blotting revealed a considerable shift in the distribution of subunits for each of the complexes with mitochondrially encoded subunits (FIGS. 3b-d, f). As expected, only complex II, which does not contain any mitochondrially-encoded subunits, failed to shift positions in the gradient (see FIG. 3e).

Western Blotting of Two-Dimensional Gels

For Western blotting of two-dimensional gels, 10-20 μl of each fraction was loaded per lane.

Using appropriate antibodies, Fractions 4 and 5 were analyzed and spots corresponding to complex V α, complex III core 2, and complex V d (all in fraction 4) could be clearly identified, as could the spot in fraction 5 corresponding to complex IV Va. Three of these proteins (complex V α, complex III core 2, and complex V d) were not identified in the human placental mitochondrial proteome of Rabilloud et al. (Electrophoresis, 19:1006-1014, 1998). This example demonstrates that the separation methods provided herein, coupled with monoclonal antibody analysis, are powerful tools in proteomics.

### Example 7

### Processing, Assembly, and Analysis of Data

This example provides a representative system used for converting raw data produced in the described separation systems into data sets that can be used to compare the protein profile of two (or more) different samples.

By way of example only, such processing includes scanning of individual two-dimensional gels, and assembling several scanned images into a three-dimensional representation of the protein profile. This can be accomplished, for instance, by sequentially stacking the individual images in an order that reflects the order of the corresponding sub-fractions in the non-denaturing fractionation (e.g., in the sucrose gradient). This is schematically illustrated in FIG. 6.

### Example 8

### Detection of Alterations in the Mitochondrial Proteome Caused by Disease

With the provision herein of enhanced methods to separate proteins from tissue, cell, or sub-cellular (e.g., organelle) samples, methods are now enabled for using the resulting proteomes to identify, diagnose, prognose, and track diseases and other clinically important conditions that alter protein expression profiles. Such alterations in protein expression profiles include changes in the amounts of individual proteins, changes in the localization of protein expression, changes in the temporal regulation of protein regulation, and particularly changes in protein-protein interactions/associations (e.g., changes in the patterns of protein complex expression).

By way of example only, the mitochondrial proteomes described above can be used to detect protein expression and association changes associated with Alzheimer's. Samples from known Alzheimer sufferer and/or a known healthy control can be separated and analyzed as described herein, to provide standard protein fingerprint(s). To determine if an individual suffers from Alzheimer's, a biological sample from that person is prepared and separated under similar or essentially identical conditions to a standard (e.g., a healthy and/or a known diseased sample). The resultant three-dimensional protein fingerprints are stained, for instance with SyproRuby™ protein gel stain as described herein, and compared to determine what proteins are increased or decreased.

It is advantageous in some instances to use computer assisted scanning and comparison procedures to produce a difference map between the two protein fingerprints. This difference map can provide qualitative and/or quantitative information regarding protein levels in the control(s) and experimental samples. In certain embodiments, proteins are identified that vary at least 20% in protein level (or level in a particular fraction, or at a particular location on a gel) between the two samples. Some proteins may vary considerably more than 20%, for instance by more than 30%, more than 40%, more than 50%, and so forth. In some instances, proteins that are present in the healthy control may be completely absent in the experimental or disease sample, and vice versa.

This disclosure provides enhanced systems for protein separation and analysis, which systems can be augmented through the use of computers and automation. Biological influences or events can be correlated with alterations in a proteome or subproteome, thus permitting disease diagnosis, prognosis, pharmaceutical agent efficacy testing, and pharmaceutical agent identification, based on observations of such alterations. It will be apparent that the precise details of the methods, products, and devices described may be varied or modified without departing from the spirit of the invention. We claim all such modifications and variations that fall within the scope and spirit of the claims below.

1. A method for creating a three-dimensional representation of a protein complement of a biological sample, comprising:
separating proteins contained in the biological sample using a non-denaturing separation process to produce a separated sample;
dividing the separated sample into a plurality of identifiable sub-fractions having an order;
subjecting at least two of the sub-fractions to at least one denaturing separation process based on protein size and at least one separation process based on protein charge, to produce a two-dimensional separation of the proteins in the sub-fractions;
producing a representation of each two-dimensional separation of proteins; and
assembling the plurality of representations in order to produce a three-dimensional representation of the proteins in the sample.

2. The method of claim 1, where the non-denaturing process comprises separation on an osmotic gradient.

3. The method of claim 2, where the osmotic gradient is a discontinuous sucrose gradient.

4. The method of claim 2, where the osmotic gradient is a continuous sucrose gradient.

5. The method of claim 1, where the denaturing separation process based on protein size comprises separation on a SDS-PAGE gel.

6. The method of claim 1, where the denaturing separation process based on protein charge comprises separation on an isoelectric focusing gel.

7. The method of claim 1, where the denaturing separation process based on protein charge is carried out before the denaturing separation process based on protein size.

8. The method of claim 1, where assembling the plurality of representations in order comprises computer processing of the representations.

9. The method of claim 1, where the biological sample comprises a sample from a plant, a fungus, an animal, or a microbial culture.

10. The method of claim 1, where the biological sample is an organellar preparation.

11. The method of claim 10, where the organellar preparation is a preparation enriched for nuclei, endoplasmic reticulum, mitochondria, plastids, lysosomes (vacuoles), peroxisomes, cytosol components, or plasma membranes.

12. The method of claim 10, where the organellar preparation is a preparation enriched for mitochondria.

13. The method of claim 1, where the biological sample is prefractionated into a plurality of pre-fractions prior to being separated into sub-fractions using a non-denaturing separation process, and where the three-dimensional representation of the proteins is a three-dimensional representation of a pre-fraction.

14. The method of claim 13, where assembling the plurality of representations in order to produce a three-dimensional representation of the proteins in the sample further comprises assembling a plurality of three-dimensional representations of individual pre-fractions into a single three-dimensional representation.

15. The method of claim 1, comprising subjecting each of the sub-fractions to at least one denaturing separation process based on protein size and at least one separation process based on protein charge, to produce a two-dimensional separation of the proteins in the sub-fractions.

16. The method of claim 1, where at least two of the sub-fractions are subject to different denaturing separation processes based on protein size and/or different denaturing separation processes based on protein charge.

17. A three-dimensional representation of a protein complement of a sample, generated by any one of the methods of claims 1-16.

18. A method for creating a three-dimensional representation of a protein complement of a mitochondrial sample, comprising:
separating proteins contained in the mitochondrial sample using a sucrose gradient to produce a separated sample;
dividing the separated sample into a plurality of identifiable sub-fractions having an order;
subjecting at least some of the sub-fractions to isoelectric gel electrophoresis, followed by SDS-PAGE, to produce a two-dimensional separation of the proteins in the sub-fractions;
producing a representation of each two-dimensional separation of proteins having a plurality of individual features; and
assembling the plurality of representations in order to produce a three-dimensional representation of the proteins in the sample.

19. The method of claim 18, further comprising identifying at least one feature in the three dimensional representation.

20. The method of claim 1, which is a method of generating a three-dimensional protein profile of a biological sample.

21. The method of claim 20, where the method is a method of generating a three-dimensional protein profile for a disease or condition, and wherein the biological sample is a sample from an organism known to be afflicted with the disease or condition.

22. The method of claim 21, where the disease or condition is linked to mitochondrial function, the three-dimensional protein profile generated is a mitochondrial disease/condition-linked profile, and the biological sample comprises an organellar preparation enriched for mitochondria.

23. A method of screening for a compound useful in treating, reducing, or preventing a disease or condition linked to mitochondrial function, or development or progression of a disease or condition linked to mitochondrial function, comprising determining if application of a test compound to a subject alters a mitochondrial disease/condition-linked profile produced from the subject, so that the profile less closely resembles a mitochondrial disease/condition-linked profile than it did prior to such treatment, and selecting a compound that so alters the profile.

24. A method of determining drug or treatment effectiveness or side effects, comprising:
applying a drug or treatment to an organism or a cell sample from the organism;
taking a biological sample from the organism or the cell sample from the organism;
analyzing the biological sample to produce a test three dimensional protein profile for the subject using the method of claim 20;
comparing the test three dimensional protein profile for the organism with a control three dimensional protein profile, which profile was generated using the method of claim 20; and
drawing conclusions about the effectiveness or side effects of the drug or treatment based on differences or similarities between the test three dimensional protein profile and the control three dimensional protein profile.

25. The method of claim 24, wherein the drug or treatment is a drug or treatment for a mitochondrial-linked disease or condition, and the test and control three dimensional protein profiles are three dimensional mitochondrial protein profiles.
